(19) United States
(12) Patent Application Publication
(10) Pub. No.: US 2010/0267637 A1
(43) Pub. Date: Oct. 21, 2010

(54) TREATMENT OF MELANOMA WITH ALPHA THYMOSIN PEPTIDES IN COMBINATION WITH A KINASE INHIBITOR

(75) Inventors: Israel Rios, Menlo Park, CA (US); Cynthia W. Tuthill, Menlo Park, CA (US)

Correspondence Address: COOLEY LLP;ATTN: Patent Group, Suite 1100, 777 - 6th Street, NW, WASHINGTON, DC, 20001, US

(73) Assignee: SciClone Pharmaceuticals, Inc., Foster City, CA (US)

(21) Appl. No.: 12/747,438

(22) PCT Filed: Dec. 12, 2008
(86) PCT No.: PCT/US08/86545

§ 371 Date: Jun. 10, 2010

Related U.S. Application Data
(60) Provisional application No. 61/013,476, filed on Dec. 13, 2007.

(51) Int. Cl. A61K 38/22; A61P 35/04; A61P 35/00

(52) U.S. Cl. 514/12.9

(57) ABSTRACT

Melanoma or a metastasis thereof is treated in a human patient in a combination therapy which includes administering a melanoma-treating combination to a human melanoma patient during a treatment regimen, the combination including an alpha thymosin peptide and a kinase inhibitor.

### CROSS-REFERENCE TO RELATED APPLICATIONS

This application claims the benefit of U.S. Provisional Application No. 61/013,476, filed Dec. 13, 2007, the disclosure of which is incorporated herein in its entirety by reference.

### FIELD OF THE INVENTION

The present invention relates to the field of melanoma treatment.

### BACKGROUND OF THE INVENTION

Skin cancer is the most common form of cancer in the United States. In 2007, The American Cancer Society estimates that approximately 8,110 deaths will occur from melanoma and another 59,940 cases of melanoma are expected to be diagnosed in this country.

Melanoma is a malignant tumor of melanocytes which are found predominantly in skin but also in bowel and the eye (uveal melanoma). It is one of the rarer types of skin cancer but causes the majority of skin cancer related deaths.

The treatment includes surgical removal of the tumor; adjuvant treatment; chemo- and immunotherapy, or radiation therapy. Of particular danger are metastases of the primary melanoma tumor.

There remains a need in the art for improved treatments of melanoma.

### SUMMARY OF THE INVENTION

In accordance with the present invention, a method of treating melanoma or a metastasis thereof in a human patient in a combination therapy which comprises administering a melanoma-treating combination to a human melanoma patient during a treatment regimen, the combination comprising an alpha thymosin peptide and a kinase inhibitor.

### DESCRIPTION OF THE PREFERRED EMBODIMENTS

The present invention is directed to a method of treating melanoma or metastases thereof in human patients. The method involves administering a melanoma-treating effective combination to human melanoma patients, the combination comprising an alpha thymosin peptide and a kinase inhibitor.

In certain embodiments, the combination further includes one or more additional agents to combat or treat melanoma.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1. Suitable dosages of the alpha thymosin peptide can be within the range of about 0.001-10 mg/kg/day.

The terms “thymosin alpha 1” and “TA1” refer to peptides having the amino acid sequence disclosed in U.S. Pat. No. 4,079,137, the disclosure of which is incorporated herein by reference.

Thymosin alpha 1 (TA1), initially isolated from Thymosin Fraction 5 (TF5), has been sequenced and chemically synthesized. TA1 is a 28 amino acid peptide with a molecular weight of 3108.

Effective amounts of an alpha thymosin peptide are amounts which may be dosage units within a range corresponding to about 0.1-20 mg of TA1, about 1-10 mg of TA1, about 2-10 mg of TA1, about 2-7 mg of TA1, or about 3-6.5 mg of TA1, and may comprise about 1.6, 3.2 or 6.4 mg of TA1, or about 3.2 or 6.4 mg of TA1. A dosage unit may be administered once per day, or a plurality of times per day.

Melanoma has various stages, which may include Stage 0, I, II, III and IV, as well as their respective subdivisions. In certain embodiments, the melanoma being treated is malignant metastatic melanoma. In certain embodiments, the melanoma being treated is stage I, stage II, stage III or stage IV. In other embodiments, the melanoma being treated is stage M1a, M1b or M1c melanoma.

The alpha thymosin peptide is administered in a treatment regimen which includes administration to the patient of a kinase inhibitor. These include, without limitation, sorafenib.

The method of the present invention comprises administering the alpha thymosin peptide along with administering a kinase inhibitor, during a course of the treatment regimen. The kinase inhibitor may be administered continuously (i.e., daily), multiple times per day, every other day, etc., and may be administered concurrently with the alpha thymosin peptide or separately therefrom during the treatment regimen, e.g., on the same day(s) as the alpha thymosin peptide or on different days during the course of the treatment regimen. In certain embodiments, the kinase inhibitor is administered in a dosage range of, e.g., about 10-2000 mg/day of administration, about 50-1000 mg/day, or about 50-800 mg/day. Daily dosages may be, e.g., about 50 mg, 100 mg, 200 mg, 300 mg, 400 mg, 500 mg, 600 mg, 700 mg, 800 mg, etc.

It surprisingly has been found that administration of an alpha thymosin peptide with a kinase inhibitor provides unexpected reduction of, and protection against, toxicity (e.g., including weight loss) to a subject, which toxicity results from administration of the kinase inhibitor to a subject.

As noted above, in certain embodiments, the combination further includes one or more additional agents to combat or treat melanoma. Such additional agents may be antineoplastic agents such as alkylating antineoplastic agents (AlkAA), which include, without limitation, dacarbazine (DTIC). Additional agent(s) of the combination, such as alkylating antineoplastic agents (AlkAA), may be administered to patient within dosage ranges of, e.g., about 700-1300 mg/m²/day, about 800-1200 mg/m²/day, or at about 1000 mg/m²/day.

The various components of the combination may be administered concurrently with, or separately from, other components in a treatment regimen.

In certain embodiments, the treatment regimen comprises a plurality of days, with the alpha thymosin peptide comprising thymosin alpha 1 (TA1), and the TA1 being administered to the patient during at least a portion of the treatment regimen at a dosage within a range of about 0.5-10 mg/day. In certain embodiments, the TA1 dosage is within a range of about 1.5-7 mg/day, or within a range of about 1.6-6.4 mg/day. In certain embodiments, the TA1 dosage is within a range of about 1.7-10 mg/day, about 1.7-7 mg/day, or about 3-7 mg/day. Exemplary dosages include about 1.6, 3.2 or 6.4 mg/day.

In certain embodiments, the treatment regimen comprises administering the alpha thymosin peptide for a period of about 1-10 days, followed by about 1-5 days of non-administration of the alpha thymosin peptide; or the alpha thymosin peptide may be administered daily for about 3-5 days, followed by about 2-4 days of non-administration of the alpha thymosin peptide. Alternatively, the alpha thymosin peptide is administered daily for about 4 days, followed by about 3 days of non-administration of the alpha thymosin peptide.

According to one embodiment, the invention comprises use of an alpha thymosin peptide and a kinase inhibitor in manufacture of a melanoma-treating effective pharmaceutical combination or medicament for use in a treatment regimen for treating melanoma or a metastasis thereof in a human melanoma patient.

According to one embodiment, said medicament is for use in a treatment regimen which substantially excludes any immune-stimulating cytokine to said patient during said treatment regimen in an amount significant for treatment of melanoma or a metastasis thereof.

According to one embodiment, said LDH blood level is below 475 IU/L.

According to one embodiment, said LDH blood level is between 100-335 IU/L.

One embodiment is the manufacture of a pharmaceutical combination including said alpha thymosin peptide, said combination further comprising a kinase inhibitor for use during a course of the treatment regimen, which alpha thymosin peptide and a kinase inhibitor may be administered separately or together.

According to one embodiment, said kinase inhibitor is sorafenib.

According to one embodiment, said medicament is for use in a treatment regimen which comprises a plurality of days, said alpha thymosin peptide comprises thymosin alpha 1 (TA1), and said TA1 is for use in administration to said patient during at least a portion of said treatment regimen at a dosage within a range of 0.5-10 mg/day.

According to one embodiment, said TA1 dosage is within a range of 1.5-7 mg/day.

According to one embodiment, said TA1 dosage is 3.2 mg/day.

According to one embodiment, said TA1 dosage is 6.4 mg/day.

According to one embodiment, said alpha thymosin peptide is TA1 and said medicament is for use in a treatment regimen which comprises administration of TA1 daily for a period of about 1-10 days, followed by about 1-5 days of non-administration of said TA1.

According to one embodiment, said TA1 is for use in administration daily for about 3-5 days, followed by about 2-4 days of non-administration of said TA1.

According to one embodiment, said TA1 is for use in administration daily for about 4 days, followed by about 3 days non-administration of said TA1.

The invention also relates to use of an alpha thymosin peptide and a kinase inhibitor in manufacture of a pharmaceutical combination for administration to a melanoma patient, wherein the alpha thymosin peptide and the kinase inhibitor may be administered separately or together, as well as to a kit comprising the alpha thymosin peptide, the kinase inhibitor, and optionally instructions for use in treatment of melanoma.

### Example 1

### Anti-Tumor Efficacy of Combined Treatment of Thymosin Alpha-1, DTIC, and Sorafenib in Mice Bearing Subcutaneous B16 Melanoma

### Abbreviations

BW Body Weight

CO₂ Carbon Dioxide

DTIC Dacarbazine

g Gram

IR Inhibition Rate

kg Kilogram

L Length

mg Milligram

mL Milliliter

NA Not Applicable

PBS Phosphate Buffered Saline

SD Standard Deviation

TA-1 Thymosin Alpha-1

TV Tumor Volume

TW Tumor Weight

W Width

Summary

In this study, the anti-tumor effect of TA-1 in combination with chemotherapeutic drugs DTIC and Sorafenib was evaluated in C57BL/6 mice bearing B16 melanoma cells. The toxic effects of the dosing regimens were also monitored. A total of 90 mice were implanted subcutaneously with murine B16 cells, followed by treatment with TA-1, Sorafenib, or DITC alone or in combination for 14 consecutive days. Meanwhile 9H10 was given in two groups via i.p. on Day 3, 6 and 9. TA-1 and DITC were administered daily by s.c. injection, Sorafenib was administered daily by p.o. In total, 9 groups were used: Group 1: vehicle; Group 2: Sorafenib 80 mg/kg; Group 3: DITC 5 mg/kg; Group 4: TA-1 6 mg/kg; Group 5: TA-1 6 mg/kg+Sorafenib 80 mg/kg; Group 6: Sorafenib 80 mg/kg+DITC 5 mg/kg; and Group 7: Sorafenib 80 mg/kg+DITC 5 mg/kg+TA-1 6 mg/kg. Tumor volume and body weight were measured every three days, and tumor weights were measured on Day 16 at the end of the study.

Tumor measurement data showed that the mean tumor volumes of all treatment except Group 3 were statistically significantly smaller than that of Group 1 on Day 12 and Day 15. On Day 16, the mean tumor weights of all treatment groups were lower than Group 1. The PI_{tw} values of Group 2, Group 3, Group 4, Group 5, Group 6, and Group 7 were 85.76%, 28.41%, 43.35%, 70.41%, 86.34%, and 84.05% respectively, indicating effectiveness of all treatment regimens.

Throughout the course of the study, there were two animal deaths noticed in Sorafenib-treated groups (Groups 6 and 7). Moreover, the mean body weights in Sorafenib treatment groups were significantly decreased. These observations suggested a strong toxic effect of Sorafenib treatment. When used alone, TA-1 did not cause any loss of body weights throughout the course of the study, indicating that TA-1 was not toxic. When TA-1 was used in combination with Sorafenib, it attenuated weight loss associated with Sorafenib treatment.

In summary, the tumor model used in this study was valid as tumor growth was inhibited by DTIC, the positive control drug. Daily administration of test article TA-1 at 6 mg/kg was effective against the tumor growth. Sorafenib treatment was also effective towards the tumor inhibition in the model. The results suggest that TA-1 may be of value for enhancing the anti-tumor effect of a chemo-therapy and/or an immunotherapy designed for melanoma patients. In addition, the combination of TA-1 with a chemo-therapy may possess an additional value in reducing the toxic adverse effect of the chemotherapy.

Introduction

Thymosin Alpha-1 (TA-1) is an immunomodulator that may possess a potential anti-tumor activity. Dacarbazine (DTIC) is the conventional chemotherapeutic drugs for the patients with melanoma. Sorafenib is an investigational drug for melanoma patients. Herein the combined anti-tumor effect of TA-1, Sorafenib, and DTIC was evaluated in the C57BL/6 mice subcutaneously implanted with B16 cells as a syngeneic melanoma model. Tumor growth was examined to explore the therapeutic potential of the combination for the treatment of melanoma. Body weights of host animals were measured to evaluate the toxic effect of the combination treatment.

Materials and Methods

Test and Control Articles

In this study, PBS was used as the negative control article (vehicle). The test article TA-1 (SciClone) was dissolved in PBS solution to achieve the proper dose concentration as indicated in Table 1. TA-1 solution was stored at 2-8° C. and used up in one week. DTIC (Sigma) was initially dissolved in 0.01 N HCl and then diluted with PBS to 1 mg/mL. DTIC dosing solution was kept on ice, protected from light, and used within one day. Sorafenib (Shanghai Yingxuan Pharmaceutical Co., LTD) was dissolved in the mixture of Cremophor EL/ethanol (50:50; Sigma Cremophor EL, 95% ethyl alcohol) at 64 mg/mL (corresponding to 4-fold of the dosing concentration, Table 1), foil wrapped, and stored at room temperature. This 4× stock solution was prepared fresh every 3 days. Final dosing solutions were prepared on the day of use by diluting the stock solution to 1× with water.

TABLE 1
Dose Formulation

| | | Dose | |
|---|---|---|---|
| | Dose level | Volume | Concentration |
| Treatment | (mg/kg) | (mL/kg) | (mg/mL) |
| TA-1 | 6 | 5 | 1.2 |
| DTIC | 5 | 5 | 1.0 |
| Sorafenib | NA | NA | 64 |
| | 80 | 5 | 16 |

Test System and Animal Husbandry

Murine B16 Melanoma Cells

Murine B16 melanoma cells were thawed from the stock of Cell Culture Center, Insitutue of Basic Medical Sciences, Peking Union Medical College and Chinese Academy of Medical Sciences (PUMC&CAMS, Beijing, P.R.China). The tumor cells were adapted in C57BL/6 mice before use in the experiment. Please refer to Section 4.3.1 for details on cell adaptation.

Test System

Forty-five male and forty-five female healthy, naive, C57BL/6 mice were received from the Institute of Laboratory Animal Science, CAMS, Beijing, P. R. China. The animals were six weeks old and weighed between 18 and 22 grams at the start of the study.

Animal Husbandry

Animals were group-housed in autoclaved shoe box cages with autoclaved wood chips as the bedding materials. The temperature of the animal room was maintained at 22 to 25° C., and the relative humidity was maintained at 40 to 60%. A 12-hour light/12-hour dark cycle was maintained except when interrupted by study-related events. Animals were fed ad libitum with sterile water and Beijing KeAoXieLi Rodent Diet (certified). All animals were acclimated for 3 days before tumor inoculation.

Experimental Procedures

Tumor Cell Adaptation

As per aseptic tissue culture procedures, one vial of B16 melanoma cells was removed from the liquid nitrogen stock, and placed into a 37° C. water bath. Gentle swirling was conducted until the content of the vial was thawed. Using tissue culture/sterile procedures, the cells were immediately centrifuged with a TD5A-WS centrifuge at 1000 rpm, 20-25° C., 5 min. After centrifugation, the cells were suspended in 0.1 to 0.5 mL normal saline (NS) and subcutaneously injected into 10 mice (0.1 mL/mouse, about 1×10⁶ cells). After 7-10 days, when the tumor diameter was approximately 1 cm, the animals were euthanized with CO₂ overdose and the tumors excised. The procedure was repeated with 20 mice to generate a sufficient number of B16 melanoma cells with adequate transplantability.

Tumor Cell Inoculation

On the day of tumor implantation, approximately 1×10⁶ cells in 0.1 mL were subcutaneously injected on the right axillary area of each mouse. The day of tumor implantation was defined as Day 0.

Study Design and Treatment Regimen

On Day 3, the animals were randomly assigned into nine different weight-matched and tumor-size-matched groups. Dosing was started on Day 3 using the regimen according to Table 2. Briefly, TA-1, DTIC, and sorafenib were administered once daily from Days 3-15.

TABLE 2
Treatment Regimen and Study Design

| Group | Group | | Number of | Dosing | Necropsy |
|---|---|---|---|---|---|
| Number | Name | Treatment | Animals | Period | Day |
| 1 | Vehicle | PBS | 10 | Days | Day 16 |
| 2 | Sorafenib | Sorafenib, 80 mg/kg, p.o., | 10 | 3-15 | |
| | | daily | | | |
| 3 | DTIC | DTIC, 5 mg/kg, s.c., daily | 10 | | |
| 4 | TA-1 | TA-1, 6 mg/kg, s.c., daily | 10 | | |
| 5 | Sorafenib + | Sorafenib, 80 mg/kg, p.o., | 10 | | |
| | TA-1 | daily + TA-1, 6 mg/kg, s.c., | | | |
| | | daily | | | |
| 6 | Sorafenib + | Sorafenib, 80 mg/kg, p.o., | 10 | | |
| | DTIC | daily + DTIC, 5 mg/kg, s.c., | | | |
| | | daily | | | |
| 7 | Sorafenib + | Sorafenib, 80 mg/kg, p.o., | 10 | | |
| | DTIC + | daily + DTIC, 5 mg/kg, s.c., | | | |
| | TA-1 | daily + TA-1, 6 mg/kg, s.c., | | | |
| | | daily | | | |

Evaluation of Anti-Tumor Effect

From Day 1 to Day 15, mortality and moribundity were checked twice daily, the body weights were recorded once every 3 days, and tumors were measured using a caliper once every 3 days. At the end of the study (Day 16), the animals were euthanized by CO₂ asphyxiation, and the tumors were excised and weighed.

Based on the tumor size, the tumor volume (TV) was calculated with the formula: [TV=(Length×Width×Width)/2]. And the percent inhibition (PI) of TV (PI_{TV}) was calculated according to the equation below:

PI_{TV}(%)=(TV vehicle−TVdrug treated)/TV vehicle×100

The anti-tumor effect of the test article was further evaluated with tumor weight (TW) measured on day of necropsy (Day 16). The PI of TW was calculated using the equation below:

PI_{TW}(%)=100×(TW vehicle−TW drug treated)/TW vehicle

The calculations of PI_{TV} and PI_{TW} were performed using an Excel spreadsheet and reviewed by the Study Director.

Evaluation of Treatment Toxicity

Toxicity of all treatment regimens was evaluated with the body weights of the study animals along with the drug-induced animal deaths. The inhibition of body weight was calculated using Excel according to the equation below:

PI_{BW}(%)=100×(BW vehicle−BW drug treated)/BW vehicle

Statistical Analysis

Inter-group comparison was performed in terms of tumor volume, tumor weight and body weight, using a student's t test. P values of less than 0.05 were considered to be statistically significant.

Results

Mortality

Throughout the course of study, there were two animal deaths. One mouse in Group 7 (Sorafenib+DITC+TA-1) died on Day 7, and another in Group 6 (Sorafenib+DITC) died on Day 13. When the deaths were noticed, there was either no measurable tumor load or fairly small tumor load in the dead animals. Moreover, significantly decreased body weights were observed before the deaths. These observations collectively suggest that the deaths were related to the drug treatment, rather as a result of tumor growth.

Tumor Size

Raw measurement data of tumor size are tabulated in Appendixes 1-10. The calculated mean tumor volumes and statistical testing results of each treatment group versus the vehicle group are tabulated in the Tables 3-7.

On Days 3 and 6 only a few mice had palpable tumors, and there was no statistical difference in tumor volume between vehicle control group and any treatment group. On Day 9, most mice of Group 1 (Vehicle Control), Group 3 (DITC) and Group 4 (TA-1), and only a few of mice in Group 2 (Sorafenib), Group 5 (Sorafenib+TA-1), tumors were recorded in Group 6 (Sorafenib+DTIC) and Group 7 (Sorafenib+DTIC+TA-1); the mean tumor volume of each group in Group 2, and Groups 5-7 was statistically significantly smaller than Group 1 (p<0.05). On Day 12 and Day 15, all surviving mice in the Groups 1-7 showed palpable tumors, and the mean tumor volume of each drug treatment group except Group 3 (DITC) was statistically significantly smaller than the vehicle control group (p<0.05).

Tumor Weight

Raw data of tumor weights measured on Day 16 are tabulated in Appendix 11. The calculated percent inhibition (PI_{tw}) values based on tumor weight and the statistical comparison results between each of the drug treatment groups and the vehicle group are tabulated in Table 8. As shown in Table 8, the mean tumor weight of each treatment group was lower than that of the vehicle group. The PI_{tw} values of Group 2, Group 3, Group 4, Group 5, Group 6 and Group 7 were 85.76%, 28.41%, 43.35%, 70.41%, 86.34% and 84.05%, respectively.

Body Weight

Raw data of body weight measurement is listed in Appendixes 12-17. The results of statistical comparison of each treatment group versus the vehicle group are tabulated in the Tables 9-14.

As shown in the Tables 9-14, there was no significant difference between each of treatment groups and the vehicle control group on Days 0, 3 and 6. On Day 9, in comparison to the vehicle group, Group 6 (Sorafenib+DITC) and Group 7 (Sorafenib+DITC+TA-1) exhibited decreases in body weight by 7.89% (p<0.05) and 7.67% (p<0.05), respectively, and there were no statistically significant differences in body weights for other groups. On Day 12, the body weights of Group 2 (Sorafenib alone), Group 6 (Sorafenib+DITC), and Group 7 (Sorafenib+DITC+TA-1) were 8.22% (p<0.05), 7.99% (p<0.05) and 11.88% (p<0.01), respectively, lower than that of the vehicle group. In contrast to the three Sorafenib-treated groups, other treatment groups did not show any differences in body weight relative to the vehicle group. The PI_{BW} values of Group 7 (Sorafenib+DITC+TA-1) was 8.71% (p<0.05) on Day 15, while the body weights of other treatment groups were not statistically significantly different from the vehicle group. These result showed that throughout the course of the study, there were no statistical differences in body weights of Group 3 (Treatment with DITC alone), Group 4 (TA-1 alone), and Group 5 (Sorafenib+TA-1). The chemo-drug Sorafenib, either it was used alone or in combination with DITC, was associated with the loss of body weight, especially when it was used in combination with DITC. Combination of Sorafenib with TA-1 seems to attenuate the Sorafenib-associated weight loss. For example, there was 8.22% (p<0.05) weight loss in Sorafenib alone group on Day 12, while the weight loss was 4.20% in Sorafenib+TA-1 group and statistically insignificant. However, combination of TA-1 with two chemo-drugs (Sorafenib+DTIC) did not reduce weight loss. This may suggest that the toxic effect of the chemo-drug combination was too strong to be attenuated by TA-1.

Conclusion and Discussion

In conclusion, the tumor model used in this study was valid as tumor growth was inhibited by positive control drug DTIC. Daily administration of test article TA-1 at 6 mg/kg was effective against the tumor growth. Throughout the course of the study, mean tumor volume in animals of Group 4 which received TA-1 treatment was significantly reduced by 50-60% in comparison to that of the vehicle control group. Tumor weights, which were measured on Day 16, were reduced by 43.35% in TA-1-treated animals. Sorafenib treatment resulted in 85.76% inhibition of tumor growth based on tumor weight measurement taken on Day 16. When Sorafenib or Sorafenib+DITC were used in combination with TA-1, there was no additional tumor inhibition. This may be due to the fact the tumor inhibition by Sorafenib was too strong and that there was little room for TA-1 to exert an additive or synergistic effect. DTIC caused a modest inhibition in tumor growth (e.g., 28.41% based on tumor weight), but the combined effect of DTIC and TA-1 was not explored in the study. When 9H10+DTIC was further combined with TA-1, the tumor inhibition rate was brought up to 62.05% (Table 8). Although these changes (e.g., 28.41% to 51.35%) were modest and not statistically significant, the observations suggest that TA-1 may be of value for enhancing anti-tumor effect of conventional chemo-therapy and/or an immunotherapy designed for melanoma patients. Refinement of test protocol (e.g., reduction of Sorafenib dose, prolongation of testing, use of survival endpoint) may help to elucidate an additive or synergistic effect of TA-1.

The mean body weights in Sorafenib treatment groups were significantly reduced. This, coupled with animal deaths observed in Sorafenib-treated groups, indicates a strong toxic effect of the chemo-treatment. When used alone, TA-1 did no cause any statistically significant loss of body weights throughout the course of the study, suggesting that TA-1 is not toxic. When TA-1 was used in combination with Sorafenib, it actually attenuated weight loss caused by Sorafenib treatment. Thus, the combined use of TA-1 with a chemo-therapy may prove an additional value.

TABLE 3
Statistical results of tumor sizes on Day 3

| | | Number | Tumor | | |
|---|---|---|---|---|---|
| Group | | of Surviving | Volume (cm³) | PI | P |
| Number | Group Name | Animals | (Mean ± SD) | (TV) | Value |
| 1 | Vehicle | 10 | 0.000 ± 0.0002 | NA | NA |
| 2 | Sorafenib | 10 | 0.001 ± 0.0017 | −750.00 | 0.1753 |
| 3 | DTIC | 10 | 0.001 ± 0.0012 | −400.00 | 0.3306 |
| 4 | TA-1 | 10 | 0.000 ± 0.0002 | 50.00 | 0.5560 |
| 5 | Sorafenib + TA-1 | 10 | 0.001 ± 0.0016 | −800.00 | 0.1449 |
| 6 | Sorafenib + DTIC | 10 | 0.000 ± 0.0002 | 50.00 | 0.5560 |
| 7 | Sorafenib + DTIC + TA-1 | 10 | 0.001 ± 0.0017 | −700.00 | 0.2092 |

TABLE 4
Statistical results of tumor sizes on Day 6

| | | Number | Tumor | | |
|---|---|---|---|---|---|
| Group | | of Surviving | Volume (cm³) | PI | P |
| Number | Group Name | Animals | (Mean ± SD) | (TV) | Value |
| 1 | Vehicle | 10 | 0.001 ± 0.0012 | NA | NA |
| 2 | Sorafenib | 10 | 0.000 ± 0.0000 | 100.00 | 0.1760 |
| 3 | DTIC | 10 | 0.000 ± 0.0002 | 90.91 | 0.2203 |
| 4 | TA-1 | 10 | 0.000 ± 0.0000 | 100.00 | 0.1760 |
| 5 | Sorafenib + TA-1 | 10 | 0.000 ± 0.0000 | 100.00 | 0.1760 |
| 6 | Sorafenib + DTIC | 10 | 0.000 ± 0.0000 | 100.00 | 0.1760 |
| 7 | Sorafenib + DTIC + TA-1 | 10 | 0.000 ± 0.0000 | 100.00 | 0.1760 |

TABLE 5
Statistical results of tumor sizes on Day 9

| | | Number | Tumor | | |
|---|---|---|---|---|---|
| Group | | of Surviving | Volume (cm³) | PI | P |
| Number | Group Name | Animals | (Mean ± SD) | (TV) | Value |
| 1 | Vehicle | 10 | 0.021 ± 0.0252 | NA | NA |
| 2 | Sorafenib | 10 | 0.000 ± 0.0002 | 99.52 | 0.0183 |
| 3 | DTIC | 10 | 0.006 ± 0.0065 | 71.15 | 0.0895 |
| 4 | TA-1 | 10 | 0.009 ± 0.0192 | 56.73 | 0.2549 |
| 5 | Sorafenib + TA-1 | 10 | 0.000 ± 0.0002 | 99.28 | 0.0186 |
| 6 | Sorafenib + DTIC | 10 | 0.000 ± 0.0000 | 100.00 | 0.0179 |
| 7 | Sorafenib + DTIC + TA-1 | 10 | 0.000 ± 0.0000 | 100.00 | 0.0246 |

TABLE 6
Statistical results of tumor sizes on Day 12

| | | Number | Tumor | | |
|---|---|---|---|---|---|
| Group | | of Surviving | Volume (cm³) | | P |
| Number | Group Name | Animals | (Mean ± SD) | PI (TV) | Value |
| 1 | Vehicle | 10 | 0.343 ± 0.2764 | NA | NA |
| 2 | Sorafenib | 10 | 0.008 ± 0.0070 | 97.64 | 0.0012 |
| 3 | DTIC | 10 | 0.195 ± 0.1932 | 43.22 | 0.1819 |
| 4 | TA-1 | 10 | 0.139 ± 0.1301 | 59.58 | 0.0488 |
| 5 | Sorafenib + TA-1 | 10 | 0.065 ± 0.0831 | 80.97 | 0.0071 |
| 6 | Sorafenib + DTIC | 10 | 0.037 ± 0.0496 | 89.22 | 0.0029 |
| 7 | Sorafenib + DTIC + TA-1 | 9 | 0.011 ± 0.0057 | 96.90 | 0.0022 |

TABLE 7
Statistical results of tumor sizes on Day 15

| | | Number | Tumor | | |
|---|---|---|---|---|---|
| Group | | of Surviving | Volume (cm³) | | P |
| Number | Group Name | Animals | (Mean ± SD) | PI (TV) | Value |
| 1 | Vehicle | 10 | 0.732 ± 0.4465 | NA | NA |
| 2 | Sorafenib | 10 | 0.054 ± 0.0399 | 92.56 | 0.0001 |
| 3 | DTIC | 10 | 0.380 ± 0.2991 | 48.12 | 0.0528 |
| 4 | TA-1 | 10 | 0.274 ± 0.1855 | 62.57 | 0.0078 |
| 5 | Sorafenib + TA-1 | 10 | 0.189 ± 0.1736 | 74.18 | 0.0021 |
| 6 | Sorafenib + DTIC | 9 | 0.082 ± 0.1095 | 88.78 | 0.0005 |
| 7 | Sorafenib + DTIC + TA-1 | 9 | 0.083 ± 0.0589 | 88.63 | 0.0005 |

TABLE 8
Statistical results of tumor weights on Day 16

| | | Number | Tumor Weight | | |
|---|---|---|---|---|---|
| Group | | of Surviving | (g) | | P |
| Number | Group Name | Animals | (Mean ± SD) | PI (TV) | Value |
| 1 | Vehicle | 10 | 1.70 ± 0.908 | NA | NA |
| 2 | Sorafenib | 10 | 0.24 ± 0.088 | 85.76 | 0.0001 |
| 3 | DTIC | 10 | 1.22 ± 0.699 | 28.41 | 0.1992 |
| 4 | TA-1 | 10 | 0.96 ± 0.688 | 43.35 | 0.0556 |
| 5 | Sorafenib + TA-1 | 10 | 0.50 ± 0.396 | 70.41 | 0.0012 |
| 6 | Sorafenib + DTIC | 9 | 0.23 ± 0.197 | 86.34 | 0.0002 |
| 7 | Sorafenib + DTIC + TA-1 | 9 | 0.27 ± 0.133 | 84.05 | 0.0002 |

TABLE 9
Statistical results of body weights on Day 0

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 20.83 ± 1.249 | NA | NA |
| 2 | Sorafenib | 10 | 21.02 ± 0.834 | −0.9121 | 0.6938 |
| 3 | DTIC | 10 | 20.86 ± 1.294 | −0.1440 | 0.9585 |
| 4 | TA-1 | 10 | 20.86 ± 1.230 | −0.1440 | 0.9574 |
| 5 | Sorafenib + TA-1 | 10 | 20.85 ± 0.910 | −0.0960 | 0.9678 |
| 6 | Sorafenib + DTIC | 10 | 20.99 ± 0.948 | −0.7681 | 0.7507 |
| 7 | Sorafenib + DTIC + TA-1 | 10 | 20.9 ± 1.361 | −0.3361 | 0.9059 |

TABLE 10
Statistical results of body weights on Day 3

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 20.88 ± 1.278 | NA | NA |
| 2 | Sorafenib | 10 | 20.88 ± 0.934 | 0.0000 | 1.0000 |
| 3 | DTIC | 10 | 21.02 ± 0.993 | −0.6705 | 0.7875 |
| 4 | TA-1 | 10 | 20.44 ± 1.460 | 2.1073 | 0.4825 |
| 5 | Sorafenib + TA-1 | 10 | 20.67 ± 0.600 | 1.0057 | 0.6437 |
| 6 | Sorafenib + DTIC | 10 | 20.81 ± 0.924 | 0.3352 | 0.8899 |
| 7 | Sorafenib + DTIC + TA-1 | 10 | 20.91 ± 1.025 | −0.1437 | 0.9545 |

TABLE 11
Statistical results of body weights on Day 6

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 20.40 ± 1.458 | NA | NA |
| 2 | Sorafenib | 10 | 20.74 ± 1.023 | −1.6667 | 0.5537 |
| 3 | DTIC | 10 | 20.41 ± 0.960 | −0.0490 | 0.9857 |
| 4 | TA-1 | 10 | 21.38 ± 1.220 | −4.8039 | 0.1205 |
| 5 | Sorafenib + TA-1 | 10 | 19.81 ± 0.809 | 2.8922 | 0.2779 |
| 6 | Sorafenib + DTIC | 10 | 19.72 ± 1.004 | 3.3333 | 0.2403 |
| 7 | Sorafenib + DTIC + TA-1 | 10 | 19.95 ± 1.302 | 2.2059 | 0.4760 |

TABLE 12
Statistical results of body weights on Day 9

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 20.41 ± 1.599 | NA | NA |
| 2 | Sorafenib | 10 | 19.75 ± 1.307 | 3.2337 | 0.3255 |
| 3 | DTIC | 10 | 20.99 ± 1.049 | −2.8417 | 0.3502 |
| 4 | TA-1 | 10 | 20.88 ± 1.111 | −2.3028 | 0.4552 |
| 5 | Sorafenib + TA-1 | 10 | 19.44 ± 0.703 | 4.7526 | 0.0960 |
| 6 | Sorafenib + DTIC | 10 | 18.80 ± 1.112 | 7.8883 | 0.0176 |
| 7 | Sorafenib + DTIC + TA-1 | 10 | 18.84 ± 1.038 | 7.6705 | 0.0230 |

TABLE 13
Statistical results of body weights on Day 12

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 21.89 ± 1.556 | NA | NA |
| 2 | Sorafenib | 10 | 20.09 ± 1.534 | 8.2229 | 0.0180 |
| 3 | DTIC | 10 | 22.09 ± 1.079 | −0.9137 | 0.7426 |
| 4 | TA-1 | 10 | 21.24 ± 1.623 | 2.9694 | 0.3732 |
| 5 | Sorafenib + TA-1 | 10 | 20.97 ± 0.865 | 4.2028 | 0.1202 |
| 6 | Sorafenib + DTIC | 10 | 20.14 ± 1.753 | 7.9945 | 0.0299 |
| 7 | Sorafenib + DTIC + TA-1 | 9 | 19.29 ± 0.895 | 11.8826 | 0.0004 |

TABLE 14
Statistical results of body weights on Day 15

| | | Number | Body Weight | | |
|---|---|---|---|---|---|
| Group | | of Surviving | (g) | PI | P |
| Number | Group Name | Animals | (Mean ± SD) | (TV) | Value |
| 1 | Vehicle | 10 | 22.14 ± 1.873 | NA | NA |
| 2 | Sorafenib | 10 | 21.41 ± 1.919 | 3.2972 | 0.4006 |
| 3 | DTIC | 10 | 22.22 ± 1.963 | −0.3613 | 0.9267 |
| 4 | TA-1 | 10 | 22.74 ± 1.459 | −2.7100 | 0.4346 |
| 5 | Sorafenib + TA-1 | 10 | 21.46 ± 0.990 | 3.0714 | 0.3235 |
| 6 | Sorafenib + DTIC | 9 | 21.71 ± 1.025 | 1.9372 | 0.5507 |
| 7 | Sorafenib + DTIC + TA-1 | 9 | 20.21 ± 1.992 | 8.7122 | 0.0440 |

APPENDIX 1
Tumor measurements (cm) on Day 3

| Group | | F1 | | F2 | | F3 | | F4 | |
|---|---|---|---|---|---|---|---|---|---|
| Number | Group Name | L | W | L | W | L | W | L | W |
| 1 | Vehicle | — | — | — | — | — | — | — | — |
| 2 | Sorafenib | — | — | 0.20 | 0.20 | 0.10 | 0.10 | — | — |
| 3 | DTIC | — | — | — | — | 0.20 | 0.20 | — | — |
| 4 | TA-1 | — | — | — | — | — | — | 0.10 | 0.10 |
| 5 | Sorafenib + TA-1 | — | — | — | — | 0.20 | 0.20 | 0.10 | 0.10 |
| 6 | Sorafenib + DTIC | — | — | — | — | — | — | — | — |
| 7 | Sorafenib + DTIC + TA-1 | 0.20 | 0.20 | — | — | — | — | — | — |

| Group | F5 | | M1 | | M2 | | M3 | | M4 | | M5 | |
|---|---|---|---|---|---|---|---|---|---|---|---|---|
| Number | L | W | L | W | L | W | L | W | L | W | L | W |
| 1 | 0.10 | 0.10 | 0.10 | 0.10 | — | — | — | — | — | — | — | — |
| 2 | — | — | 0.20 | 0.20 | — | — | — | — | — | — | — | — |
| 3 | — | — | — | — | 0.10 | 0.10 | — | — | — | — | 0.10 | 0.10 |
| 4 | — | — | — | — | — | — | — | — | — | — | — | — |
| 5 | 0.10 | 0.10 | — | — | — | — | — | — | — | — | 0.20 | 0.20 |
| 6 | 0.10 | 0.10 | — | — | — | — | — | — | — | — | — | — |
| 7 | 0.20 | 0.20 | — | — | — | — | — | — | — | — | — | — |

Note:
The sign “—” indicates that tumor does not reach a measurable size.

APPENDIX 2
Tumor volumes* (cm³) on Day 3

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle | 0.0000 | 0.0000 | 0.0000 | 0.0000 | 0.0005 | 0.0005 | 0.0000 | 0.0000 | 0.0000 | 0.0000 |
| 2 | Sorafenib | 0.0000 | 0.0040 | 0.0005 | 0.0000 | 0.0000 | 0.0040 | 0.0000 | 0.0000 | 0.0000 | 0.0000 |
| 3 | DTIC | 0.0000 | 0.0000 | 0.0040 | 0.0000 | 0.0000 | 0.0000 | 0.0005 | 0.0000 | 0.0000 | 0.0005 |
| 4 | TA-1 | 0.0000 | 0.0000 | 0.0000 | 0.0005 | 0.0000 | 0.0000 | 0.0000 | 0.0000 | 0.0000 | 0.0000 |
| 5 | Sorafenib + TA-1 | 0.0000 | 0.0000 | 0.0040 | 0.0005 | 0.0005 | 0.0000 | 0.0000 | 0.0000 | 0.0000 | 0.0040 |
| 6 | Sorafenib + DTIC | 0.0000 | 0.0000 | 0.0000 | 0.0000 | 0.0005 | 0.0000 | 0.0000 | 0.0000 | 0.0000 | 0.0000 |
| 7 | Sorafenib + DTIC + TA-1 | 0.0040 | 0.0000 | 0.0000 | 0.0000 | 0.0040 | 0.0000 | 0.0000 | 0.0000 | 0.0000 | 0.0000 |

*Tumor volumes were calculated using the formula “Tumor Volume = Length × Width × Width/2” based on the data listed in Appendix 1.

APPENDIX 3
Tumor measurements (cm) on Day 6

| Group | | F1 | | F2 | | F3 | | F4 | | F5 | | M1 | | M2 | | M3 | | M4 | | M5 | |
|---|---|---|---|---|---|---|---|---|---|---|---|---|---|---|---|---|---|---|---|---|---|
| Number | Group Name | L | W | L | W | L | W | L | W | L | W | L | W | L | W | L | W | L | W | L | W |
| 1 | Vehicle | 0.10 | 0.10 | — | — | — | — | — | — | — | — | — | — | — | — | 0.10 | 0.10 | 0.20 | 0.20 | 0.10 | 0.10 |
| 2 | Sorafenib | — | — | — | — | — | — | — | — | — | — | — | — | — | — | — | — | — | — | — | — |
| 3 | DTIC | — | — | — | — | 0.10 | 0.10 | — | — | — | — | — | — | — | — | — | — | — | — | — | — |
| 4 | TA-1 | — | — | — | — | — | — | — | — | — | — | — | — | — | — | — | — | — | — | — | — |
| 5 | Sorafenib + TA-1 | — | — | — | — | — | — | — | — | — | — | — | — | — | — | — | — | — | — | — | — |
| 6 | Sorafenib + DTIC | — | — | — | — | — | — | — | — | — | — | — | — | — | — | — | — | — | — | — | — |
| 7 | Sorafenib + DTIC + | — | — | — | — | — | — | — | — | — | — | — | — | — | — | — | — | — | — | — | — |
| | TA-1 | | | | | | | | | | | | | | | | | | | | |

Note:
The sign “—” indicates that tumor does not reach a measurable size.

APPENDIX 4
Tumor volumes* (cm³) on Day 6

| Group | | | | | | | | | | | |
|---|---|---|---|---|---|---|---|---|---|---|---|
| Number | Group Name | F1 | F2 | F3 | F4 | F5 | M1 | M2 | M3 | M4 | M5 |
| 1 | Vehicle | 0.0005 | 0.0000 | 0.0000 | 0.0000 | 0.0000 | 0.0000 | 0.0000 | 0.0005 | 0.0040 | 0.0005 |
| 2 | Sorafenib | 0.0000 | 0.0000 | 0.0000 | 0.0000 | 0.0000 | 0.0000 | 0.0000 | 0.0000 | 0.0000 | 0.0000 |
| 3 | DTIC | 0.0000 | 0.0000 | 0.0005 | 0.0000 | 0.0000 | 0.0000 | 0.0000 | 0.0000 | 0.0000 | 0.0000 |
| 4 | TA-1 | 0.0000 | 0.0000 | 0.0000 | 0.0000 | 0.0000 | 0.0000 | 0.0000 | 0.0000 | 0.0000 | 0.0000 |
| 5 | Sorafenib + TA-1 | 0.0000 | 0.0000 | 0.0000 | 0.0000 | 0.0000 | 0.0000 | 0.0000 | 0.0000 | 0.0000 | 0.0000 |
| 6 | Sorafenib + DTIC | 0.0000 | 0.0000 | 0.0000 | 0.0000 | 0.0000 | 0.0000 | 0.0000 | 0.0000 | 0.0000 | 0.0000 |
| 7 | Sorafenib + DTIC + TA-1 | 0.0000 | 0.0000 | 0.0000 | 0.0000 | 0.0000 | 0.0000 | 0.0000 | 0.0000 | 0.0000 | 0.0000 |

*Tumor volumes were calculated using the formula “Tumor Volume = Length × Width × Width/2″ based on the data listed in Appendix 3.

APPENDIX 5
Tumor measurements (cm) on Day 9

| Group | | | | | F1 | | F2 | | F3 | | F4 | |
|---|---|---|---|---|---|---|---|---|---|---|---|---|
| Number | Group Name | | | | L | W | L | W | L | W | L | W |
| 1 | Vehicle | | | | 0.40 | 0.40 | 0.10 | 0.10 | 0.10 | 0.10 | 0.20 | 0.20 |
| 2 | Sorafenib | | | | — | — | — | — | — | — | — | — |
| 3 | DTIC | | | | 0.20 | 0.20 | — | — | 0.10 | 0.10 | 0.10 | 0.10 |
| 4 | TA-1 | | | | 0.10 | 0.10 | 0.10 | 0.10 | 0.10 | 0.10 | 0.20 | 0.20 |
| 5 | Sorafenib + TA-1 | | | | — | — | 0.10 | 0.10 | — | — | 0.10 | 0.10 |
| 6 | Sorafenib + DTIC | | | | — | — | — | — | — | — | — | — |
| 7 | Sorafenib + DTIC + TA-1 | | | | — | — | — | — | — | — | — | — |
| Group | F5 | | M1 | | M2 | | M3 | | M4 | | M5 | |
| Number | L | W | L | W | L | W | L | W | L | W | L | W |
| 1 | — | — | 0.10 | 0.10 | 0.50 | 0.50 | 0.30 | 0.30 | 0.50 | 0.50 | 0.40 | 0.40 |
| 2 | 0.10 | 0.10 | — | — | — | — | — | — | — | — | 0.10 | 0.10 |
| 3 | 0.10 | 0.10 | 0.30 | 0.30 | 0.30 | 0.30 | 0.30 | 0.30 | 0.30 | 0.30 | 0.10 | 0.10 |
| 4 | — | — | 0.10 | 0.10 | 0.20 | 0.20 | 0.50 | 0.50 | 0.20 | 0.20 | 0.30 | 0.30 |
| 5 | — | — | — | — | 0.10 | 0.10 | — | — | — | — | — | — |
| 6 | — | — | — | — | — | — | — | — | — | — | — | — |
| 7 | — | — | — | — | / | / | — | — | — | — | — | — |

Note:
The sign “—” indicates that tumor does not reach a measurable size, while the sign “/” indicates a dead animal.

APPENDIX 6
Tumor volumes* (cm³) on Day 9

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 0.0320 | 0.0005 | 0.0005 | 0.0040 | 0.0000 | 0.0005 | 0.0625 | 0.0135 | 0.0625 | 0.0320 |
| 2 | Sorafenib | 0.0000 | 0.0000 | 0.0000 | 0.0000 | 0.0005 | 0.0000 | 0.0000 | 0.0000 | 0.0000 | 0.0005 |
| 3 | DTIC | 0.0040 | 0.0000 | 0.0005 | 0.0005 | 0.0005 | 0.0135 | 0.0135 | 0.0135 | 0.0135 | 0.0005 |
| 4 | TA-1 | 0.0005 | 0.0005 | 0.0005 | 0.0040 | 0.0000 | 0.0005 | 0.0040 | 0.0625 | 0.0040 | 0.0135 |
| 5 | Sorafenib + TA-1 | 0.0000 | 0.0005 | 0.0000 | 0.0005 | 0.0000 | 0.0000 | 0.0005 | 0.0000 | 0.0000 | 0.0000 |
| 6 | Sorafenib + DTIC | 0.0000 | 0.0000 | 0.0000 | 0.0000 | 0.0000 | 0.0000 | 0.0000 | 0.0000 | 0.0000 | 0.0000 |
| 7 | Sorafenib + DTIC + TA-1 | 0.0000 | 0.0000 | 0.0000 | 0.0000 | 0.0000 | 0.0000 | / | 0.0000 | 0.0000 | 0.0000 |

*Tumor volumes were calculated using the formula “Tumor Volume = Length × Width × Width/2″ based on the data listed in Appendix 5.

APPENDIX 7
Tumor measurements (cm) on Day 12

| Group | | | | | F1 | | F2 | | F3 | | F4 | |
|---|---|---|---|---|---|---|---|---|---|---|---|---|
| Number | Group Name | | | | L | W | L | W | L | W | L | W |
| 1 | Vehicle | | | | 1.00 | 1.00 | 0.40 | 0.40 | 0.50 | 0.50 | 1.00 | 1.00 |
| 2 | Sorafenib | | | | 0.30 | 0.30 | — | — | 0.30 | 0.30 | 0.30 | 0.30 |
| 3 | DTIC | | | | 0.50 | 0.50 | 0.30 | 0.30 | 0.30 | 0.30 | 0.70 | 0.70 |
| 4 | TA-1 | | | | 0.50 | 0.50 | 0.50 | 0.50 | 0.50 | 0.50 | 0.70 | 0.70 |
| 5 | Sorafenib + TA-1 | | | | 0.30 | 0.30 | 0.70 | 0.70 | 0.40 | 0.40 | 0.80 | 0.80 |
| 6 | Sorafenib + DTIC | | | | 0.40 | 0.40 | 0.30 | 0.30 | — | — | 0.30 | 0.30 |
| 7 | Sorafenib + DTIC + TA-1 | | | | 0.30 | 0.30 | 0.30 | 0.30 | 0.30 | 0.30 | 0.30 | 0.30 |
| Group | F5 | | M1 | | M2 | | M3 | | M4 | | M5 | |
| Number | L | W | L | W | L | W | L | W | L | W | L | W |
| 1 | 0.30 | 0.30 | 0.50 | 0.50 | 1.10 | 1.10 | 0.90 | 0.90 | 1.20 | 1.10 | 1.00 | 1.00 |
| 2 | 0.30 | 0.30 | — | — | 0.30 | 0.30 | — | — | — | — | 0.30 | 0.30 |
| 3 | 3.00 | 0.30 | 0.70 | 0.70 | 0.90 | 0.90 | 1.00 | 1.00 | 1.00 | 1.00 | 0.30 | 0.30 |
| 4 | 0.10 | 0.10 | 0.50 | 0.50 | 0.70 | 0.70 | 0.90 | 0.90 | 0.50 | 0.50 | 0.90 | 0.90 |
| 5 | 0.50 | 0.50 | 0.30 | 0.30 | 0.50 | 0.50 | 0.30 | 0.30 | 0.30 | 0.30 | 0.30 | 0.30 |
| 6 | 0.30 | 0.30 | 3.30 | 0.30 | 0.30 | 0.30 | 0.30 | 0.30 | 0.30 | 0.30 | 0.60 | 0.60 |
| 7 | 0.30 | 0.30 | 0.10 | 0.10 | / | / | 0.30 | 0.30 | 0.10 | 0.10 | 0.30 | 0.30 |

Note:
The sign “—” indicates that tumor does not reach a measurable size, while the sign “/” indicates a dead animal.

APPENDIX 8
Tumor volumes* (cm³) on Day 12

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 0.5000 | 0.0320 | 0.0625 | 0.5000 | 0.0135 | 0.0625 | 0.6655 | 0.3645 | 0.7260 | 0.5000 |
| 2 | Sorafenib | 0.0135 | 0.0000 | 0.0135 | 0.0135 | 0.0135 | 0.0000 | 0.0135 | 0.0000 | 0.0000 | 0.0135 |
| 3 | DTIC | 0.0625 | 0.0135 | 0.0135 | 0.1715 | 0.1350 | 0.1715 | 0.3645 | 0.5000 | 0.5000 | 0.0135 |
| 4 | TA-1 | 0.0625 | 0.0625 | 0.0625 | 0.1715 | 0.0005 | 0.0625 | 0.1715 | 0.3645 | 0.0625 | 0.3645 |
| 5 | Sorafenib + TA-1 | 0.0135 | 0.1715 | 0.0320 | 0.2560 | 0.0625 | 0.0135 | 0.0625 | 0.0135 | 0.0135 | 0.0135 |
| 6 | Sorafenib + DTIC | 0.0320 | 0.0135 | 0.0000 | 0.0135 | 0.0135 | 0.1485 | 0.0135 | 0.0135 | 0.0135 | 0.1080 |
| 7 | Sorafenib + DTIC + TA-1 | 0.0135 | 0.0135 | 0.0135 | 0.0135 | 0.0135 | 0.0005 | / | 0.0135 | 0.0005 | 0.0135 |

*Tumor volumes were calculated using the formula “Tumor Volume = Length × Width × Width/2″ based on the data listed in Appendix 7.

APPENDIX 9
Tumor measurements (cm) on Day 15

| Group | | | | | F1 | | F2 | | F3 | | F4 | |
|---|---|---|---|---|---|---|---|---|---|---|---|---|
| Number | Group Name | | | | L | W | L | W | L | W | L | W |
| 1 | Vehicle | | | | 1.20 | 1.20 | 0.80 | 0.80 | 0.80 | 0.80 | 1.30 | 1.30 |
| 2 | Sorafenib | | | | 0.60 | 0.60 | 0.40 | 0.40 | 0.60 | 0.60 | 0.70 | 0.50 |
| 3 | DTIC | | | | 0.80 | 0.80 | 0.50 | 0.50 | 0.60 | 0.60 | 0.70 | 0.70 |
| 4 | TA-1 | | | | 0.70 | 0.70 | 0.70 | 0.60 | 0.60 | 0.60 | 1.00 | 0.70 |
| 5 | Sorafenib + TA-1 | | | | 0.40 | 0.40 | 0.80 | 0.80 | 0.60 | 0.60 | 1.00 | 1.00 |
| 6 | Sorafenib + DTIC | | | | 0.50 | 0.50 | / | / | 0.30 | 0.30 | 0.40 | 0.40 |
| 7 | Sorafenib + DTIC + TA-1 | | | | 0.40 | 0.40 | 0.40 | 0.40 | 0.60 | 0.60 | 0.70 | 0.70 |
| Group | F5 | | M1 | | M2 | | M3 | | M4 | | M5 | |
| Number | L | W | L | W | L | W | L | W | L | W | L | W |
| 1 | 0.70 | 0.70 | 0.80 | 0.80 | 1.30 | 1.30 | 1.20 | 1.20 | 1.40 | 1.30 | 1.30 | 1.40 |
| 2 | 0.50 | 0.50 | 0.20 | 0.20 | 0.50 | 0.50 | 0.20 | 0.20 | 0.30 | 0.30 | 0.50 | 0.50 |
| 3 | 0.60 | 0.60 | 1.00 | 1.00 | 1.20 | 1.20 | 1.00 | 1.00 | 1.20 | 1.20 | 0.90 | 0.90 |
| 4 | 0.30 | 0.30 | 0.70 | 0.70 | 1.00 | 1.00 | 1.00 | 1.00 | 1.00 | 0.90 | 1.00 | 1.00 |
| 5 | 0.60 | 0.60 | 0.60 | 0.60 | 1.00 | 1.00 | 0.60 | 0.60 | 0.50 | 0.50 | 0.60 | 0.60 |
| 6 | 0.50 | 0.50 | 0.40 | 0.40 | 0.40 | 0.40 | 0.40 | 0.40 | 0.60 | 0.60 | 0.90 | 0.90 |
| 7 | 0.60 | 0.60 | 0.40 | 0.40 | / | / | 0.50 | 0.50 | 0.40 | 0.40 | 0.70 | 0.70 |

Note:
The sign “/” indicates a dead animal.

APPENDIX 10
Tumor volumes* (cm³) on Day 15

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 0.8640 | 0.2560 | 0.2560 | 1.0985 | 0.1715 | 0.2560 | 1.0985 | 0.8640 | 1.1830 | 1.2740 |
| 2 | Sorafenib | 0.1080 | 0.0320 | 0.1080 | 0.0875 | 0.0625 | 0.0040 | 0.0625 | 0.0040 | 0.0135 | 0.0625 |
| 3 | DTIC | 0.2560 | 0.0625 | 0.1080 | 0.1715 | 0.1080 | 0.5000 | 0.8640 | 0.5000 | 0.8640 | 0.3645 |
| 4 | TA-1 | 0.1715 | 0.1260 | 0.1080 | 0.2450 | 0.0135 | 0.1715 | 0.5000 | 0.5000 | 0.4050 | 0.5000 |
| 5 | Sorafenib + TA-1 | 0.0320 | 0.2560 | 0.1080 | 0.5000 | 0.1080 | 0.1080 | 0.5000 | 0.1080 | 0.0625 | 0.1080 |
| 6 | Sorafenib + DTIC | 0.0625 | / | 0.0135 | 0.0320 | 0.0625 | 0.0320 | 0.0320 | 0.0320 | 0.1080 | 0.3645 |
| 7 | Sorafenib + DTIC + TA-1 | 0.0320 | 0.0320 | 0.1080 | 0.1715 | 0.1080 | 0.0320 | / | 0.0625 | 0.0320 | 0.1715 |

*Tumor volumes were calculated using the formula “Tumor Volume = Length × Width × Width/2″ based on the data listed in Appendix 9.

APPENDIX 11
Tumor weight* (g) on Day 16

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 1.90 | 0.76 | 0.68 | 2.42 | 0.72 | 0.67 | 2.50 | 1.85 | 2.60 | 2.90 |
| 2 | Sorafenib | 0.23 | 0.16 | 0.32 | 0.30 | 0.16 | 0.10 | 0.38 | 0.20 | 0.25 | 0.32 |
| 3 | DTIC | 1.40 | 0.43 | 0.47 | 0.68 | 0.56 | 1.80 | 1.80 | 0.83 | 2.30 | 1.90 |
| 4 | TA-1 | 0.63 | 0.44 | 0.45 | 0.75 | 0.08 | 0.73 | 1.30 | 2.00 | 2.20 | 1.05 |
| 5 | Sorafenib + TA-1 | 0.14 | 0.58 | 0.33 | 1.24 | 0.37 | 0.25 | 1.20 | 0.36 | 0.20 | 0.36 |
| 6 | Sorafenib + DTIC | 0.14 | / | 0.00 | 0.21 | 0.19 | 0.09 | 0.20 | 0.26 | 0.30 | 0.70 |
| 7 | Sorafenib + DTIC + TA-1 | 0.16 | 0.13 | 0.38 | 0.55 | 0.29 | 0.14 | / | 0.29 | 0.26 | 0.24 |

Note:
The sign “/” indicates a dead animal.

APPENDIX 12
Body weights (g) on Day 0

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 20.70 | 21.30 | 19.70 | 20.00 | 20.00 | 20.00 | 19.90 | 20.80 | 23.50 | 22.40 |
| 2 | Sorafenib | 20.50 | 20.40 | 20.60 | 21.00 | 20.70 | 22.70 | 22.10 | 21.00 | 21.30 | 19.90 |
| 3 | DTIC | 21.50 | 19.90 | 19.70 | 19.30 | 22.10 | 22.40 | 21.80 | 21.00 | 18.90 | 22.00 |
| 4 | TA-1 | 21.40 | 19.20 | 21.10 | 20.20 | 19.00 | 21.60 | 22.50 | 22.10 | 21.70 | 19.80 |
| 5 | Sorafenib + TA-1 | 21.00 | 19.40 | 20.50 | 20.80 | 20.50 | 21.80 | 22.00 | 21.20 | 19.50 | 21.80 |
| 6 | Sorafenib + DTIC | 20.90 | 20.90 | 21.20 | 20.90 | 19.90 | 20.00 | 21.80 | 21.70 | 22.80 | 19.80 |
| 7 | Sorafenib + DTIC + TA-1 | 19.60 | 19.50 | 21.90 | 21.40 | 20.00 | 20.00 | 20.20 | 20.30 | 22.90 | 23.20 |

APPENDIX 13
Body weights (g) on Day 3

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 21.60 | 20.60 | 20.00 | 19.10 | 20.50 | 19.70 | 20.80 | 20.80 | 23.50 | 22.20 |
| 2 | Sorafenib | 20.20 | 20.50 | 20.80 | 20.40 | 20.80 | 22.10 | 22.70 | 20.90 | 21.00 | 19.40 |
| 3 | DTIC | 21.60 | 19.60 | 20.50 | 20.30 | 21.30 | 22.20 | 22.00 | 21.20 | 19.50 | 22.00 |
| 4 | TA-1 | 20.90 | 19.40 | 20.40 | 20.30 | 17.20 | 21.40 | 22.20 | 21.60 | 21.50 | 19.50 |
| 5 | Sorafenib + TA-1 | 20.90 | 20.30 | 20.00 | 20.70 | 20.10 | 21.40 | 21.50 | 20.40 | 20.00 | 21.40 |
| 6 | Sorafenib + DTIC | 21.50 | 20.50 | 20.40 | 20.30 | 19.10 | 21.00 | 21.00 | 21.70 | 22.40 | 20.20 |
| 7 | Sorafenib + DTIC + TA-1 | 20.20 | 20.50 | 21.40 | 20.70 | 20.30 | 19.60 | 20.70 | 20.40 | 22.50 | 22.80 |

APPENDIX 14
Body weights (g) on Day 6

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 21.30 | 19.50 | 19.10 | 18.90 | 19.30 | 18.70 | 21.50 | 21.30 | 22.90 | 21.50 |
| 2 | Sorafenib | 19.60 | 19.90 | 20.90 | 20.30 | 20.90 | 21.40 | 23.00 | 20.90 | 21.00 | 19.50 |
| 3 | DTIC | 20.40 | 18.40 | 19.70 | 20.10 | 20.50 | 20.90 | 21.80 | 20.20 | 20.50 | 21.60 |
| 4 | TA-1 | 22.10 | 20.10 | 21.80 | 20.80 | 20.20 | 22.00 | 22.90 | 22.30 | 22.40 | 19.20 |
| 5 | Sorafenib + TA-1 | 19.40 | 19.20 | 18.90 | 19.40 | 19.50 | 21.40 | 20.40 | 19.50 | 19.50 | 20.90 |
| 6 | Sorafenib + DTIC | 19.70 | 19.40 | 19.20 | 19.10 | 17.70 | 20.10 | 20.10 | 20.30 | 21.60 | 20.00 |
| 7 | Sorafenib + DTIC + TA-1 | 20.40 | 20.20 | 18.70 | 20.00 | 19.80 | 20.00 | 17.30 | 20.20 | 22.40 | 20.50 |

APPENDIX 15
Body weights (g) on Day 9

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 21.40 | 19.70 | 18.20 | 20.00 | 19.50 | 18.30 | 20.70 | 21.50 | 23.40 | 21.40 |
| 2 | Sorafenib | 18.60 | 18.40 | 18.40 | 18.60 | 19.80 | 20.80 | 22.10 | 20.40 | 21.10 | 19.30 |
| 3 | DTIC | 21.70 | 19.50 | 21.10 | 19.50 | 21.70 | 21.30 | 22.10 | 20.60 | 20.00 | 22.40 |
| 4 | TA-1 | 21.50 | 19.70 | 20.30 | 20.40 | 19.20 | 21.50 | 22.40 | 22.00 | 21.90 | 19.90 |
| 5 | Sorafenib + TA-1 | 18.00 | 19.20 | 19.30 | 19.50 | 19.70 | 19.50 | 20.60 | 19.50 | 18.90 | 20.20 |
| 6 | Sorafenib + DTIC | 18.60 | 17.80 | 19.60 | 18.60 | 16.50 | 20.50 | 19.50 | 18.50 | 19.60 | 18.80 |
| 7 | Sorafenib + DTIC + TA-1 | 18.50 | 19.10 | 17.80 | 19.10 | 18.30 | 19.80 | / | 18.00 | 21.00 | 18.00 |

Note
The sign “/” indicates a dead animal.

APPENDIX 16
Body weights (g) on Day 12

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 22.90 | 22.20 | 20.50 | 20.40 | 21.60 | 19.30 | 21.60 | 22.90 | 24.70 | 22.80 |
| 2 | Sorafenib | 19.00 | 17.20 | 20.80 | 18.30 | 20.30 | 22.10 | 21.40 | 20.40 | 21.50 | 19.90 |
| 3 | DTIC | 22.50 | 20.10 | 21.40 | 21.70 | 22.20 | 22.80 | 23.40 | 22.20 | 21.00 | 23.60 |
| 4 | TA-1 | 21.90 | 19.10 | 20.70 | 20.90 | 18.90 | 22.60 | 23.50 | 22.70 | 22.40 | 19.70 |
| 5 | Sorafenib + TA-1 | 21.20 | 20.50 | 20.40 | 20.20 | 21.00 | 19.80 | 22.40 | 21.70 | 20.40 | 22.10 |
| 6 | Sorafenib + DTIC | 20.80 | 16.30 | 21.10 | 20.30 | 18.10 | 22.00 | 21.20 | 21.00 | 21.30 | 19.30 |
| 7 | Sorafenib + DTIC + TA-1 | 19.00 | 20.00 | 20.10 | 20.00 | 18.60 | 20.30 | / | 17.70 | 18.50 | 19.40 |

Note:
The sign “/” indicates a dead animal.

APPENDIX 17
Body weights (g) on Day 15

| Group Number | Group Name | F1 | F2 | F3 | F4 | F5 | M1 | M2 | M3 | M4 | M5 |
|---|---|---|---|---|---|---|---|---|---|---|---|
| 1 | Vehicle | 21.40 | 22.50 | 19.50 | 21.50 | 21.40 | 20.00 | 21.80 | 23.60 | 25.70 | 24.00 |
| 2 | Sorafenib | 20.70 | 17.50 | 21.10 | 19.90 | 21.50 | 23.90 | 23.80 | 22.20 | 22.80 | 20.70 |
| 3 | DTIC | 21.30 | 18.70 | 20.20 | 22.20 | 22.20 | 24.50 | 24.20 | 22.70 | 21.30 | 24.90 |
| 4 | TA-1 | 24.30 | 20.70 | 22.30 | 21.80 | 21.30 | 22.60 | 24.50 | 24.70 | 23.70 | 21.50 |
| 5 | Sorafenib + TA-1 | 21.80 | 19.30 | 21.20 | 21.30 | 21.20 | 21.70 | 22.70 | 22.10 | 20.70 | 22.60 |
| 6 | Sorafenib + DTIC | 21.80 | / | 22.20 | 20.80 | 19.70 | 22.40 | 22.80 | 22.50 | 22.30 | 20.90 |
| 7 | Sorafenib + DTIC + TA-1 | 20.80 | 20.90 | 21.30 | 21.40 | 20.00 | 20.70 | / | 18.80 | 15.60 | 22.40 |

Note:
The sign “/” indicates a dead animal.

### Example 2

In this regimen, TA1 is administered to melanoma patients in a treatment regimen at a dosage within a range of 0.5-10 mg/day.

The melanoma patients also are treated with sorafenib at a dose level of 400 mg twice daily.

1. A method of treating melanoma or a metastasis thereof in a human patient in a combination therapy which comprises administering a melanoma-treating effective combination to a human melanoma patient during a treatment regimen, the combination comprising an alpha thymosin peptide and a kinase inhibitor.

2. The method of claim 1 wherein said kinase inhibitor comprises sorafenib.

3. The method of claim 1 wherein said treatment regimen comprises a plurality of days, said alpha thymosin peptide comprises thymosin alpha 1 (TA1), and said TA1 is administered to said patient during at least a portion of said treatment regimen at a dosage within a range of about 0.5-10 mg/day.

4. The method of claim 3 wherein said dosage is within a range of about 1.5-7 mg/day.

5. The method of claim 3 wherein said dosage is within a range of about 3-7 mg/day.

6. The method of claim 3 wherein said dosage is about 3.2 mg/day.

7. The method of claim 3 wherein said dosage is about 6.4 mg/day.

8. The method of claim 1 wherein said alpha thymosin peptide is TA1 and said treatment regimen comprises administration of TA1 daily for a period of about 1-10 days, followed by about 1-5 days of non-administration of said TA1.

9. The method of claim 8 wherein said TA1 is administered daily for about 3-5 days, followed by about 2-4 days of non-administration of said TA1.

10. The method of claim 8 wherein said TA1 is administered daily for about 4 days, followed by about 3 days non-administration of said TA1.

11. The method of claim 1 wherein said kinase inhibitor is administered to said patient at a dosage within a range of about 10-2000 mg/day.

12. The method of claim 1 wherein said kinase inhibitor is administered to said patient at a dosage of about 50-800 mg/day.

13. The method of claim 1, wherein said combination further includes administration of an alkylating antineoplastic agent (AlkAA).

14. The method of claim 13 wherein the alkylating antineoplastic agent (AlkAA) comprises dacarbazine (DTIC).

15. The method of claim 13 wherein the alkylating antineoplastic agent (AlkAA) is administered to said patient at a dosage within a range of about 700-1300 mg/m²/day.

16. The method of claim 13 wherein the alkylating antineoplastic agent (AlkAA) is administered to said patient at a dosage within a range of about 800-1200 mg/m²/day.

17. The method of claim 1 wherein said alpha thymosin peptide reduces toxicity of said kinase inhibitor in said patient.

18. The method of claim 17 wherein said toxicity includes weight loss in said patient.
